(19) United States
(12) Patent Application Publication
(10) Pub. No.: US 2026/0331275 A1
(43) Pub. Date: Oct. 22, 2026

(54) METHOD AND APPARATUS FOR PERFORMING IMAGE-GUIDED INTERVENTIONS WITH AN AERIAL 3D DISPLAY

(71) Applicant: Irving N. WEINBERG, Chevy Chase, MD (US)

(72) Inventor: Irving N. WEINBERG, Chevy Chase, MD (US)

(21) Appl. No.: 19/650,796

(22) Filed: Apr. 17, 2026

Related U.S. Application Data
(60) Provisional application No. 63/791,054, filed on Apr. 18, 2025.

(51) Int. Cl. G16H 40/63; A61N 2/00; G16H 20/30; H04N 13/117; H04N 13/366

(52) U.S. Cl. CPC G16H 40/63; A61N 2/006; G16H 20/30; H04N 13/117; H04N 13/366

(57) ABSTRACT

An apparatus and method are provided for user interaction with an aerial holographic display. A computer and display may emit light to generate the perceived image relative to a human viewer. Information is collected by one or more cameras and used to capture a position or orientation of the at least one marker, manipulatable by the human viewer, with respect to an object in the perceived 3D image

### CROSS-REFERENCE AND PRIORITY CLAIM

This application claims priority to U.S. Provisional Patent Application Ser. No. 63/791,054, entitled “METHOD AND APPARATUS FOR PERFORMING IMAGE-GUIDED INTERVENTIONS WITH AN AERIAL 3D DISPLAY” filed Apr. 18, 2025, the entirety of which is incorporated by reference.

### BACKGROUND

Aerial “floating” three-dimensional (3D) displays use lenses or mirrors to angle light from a monitor into viewers'eyes, creating disparity images that when viewed by each eye form an illusion of a three-dimensional perceived object in space, as taught by Y Sato et al in the 2021 Applied Optics article entitled “Aerial floating 3D display device with lenticular-type naked-eye 3D display and a crossed-mirror array” (DOI:10.1364/AO.436060). As taught by Sato, the viewers eye position can be used to modify the disparity images to take into account head position. Users of image-guided applications (e.g., surgeons engaged in image-guided therapy) sometimes need to localize a position or region in the same 3D space for an intervention. As taught in the SPIE Medical Imaging 2015 Proceedings by S. Anishchenko et al, entitled “Evaluation of a video-based head motion tracking system for dedicated brain PET”, (DOI: 10.1117/12.2082137), it is possible to mount cameras to record the orientation of a human head. A two-dimensional aerial projection of a touchscreen has been used to record preferences of a user, as taught by M. Otsubo in Volume 28 of the 2021 Proceedings of the International Display Workshops entitled “Aerial Imaging Principle and its Commercialization and Future Developments”. However, the ability of a user to interact with a 3D aerial object is limited.

### SUMMARY

Disclosed embodiments describe an apparatus and method for user interaction with an aerial holographic display.

In some embodiments, an apparatus for image-guided intervention comprises one or more cameras configured to capture images of a human viewer, components for generating an aerial perceived 3D image of an actual object, and at least one marker visible to the one or more cameras. Information collected by one or more cameras may used to capture a position or orientation of the at least one marker, manipulatable by the human viewer, with respect to the object in the perceived 3D image.

In some embodiments, method of image-guided intervention comprises generating an aerial perceived 3D image of an actual object based on images of a user captured by an apparatus with one or more cameras, placing a marker within the perceived 3D image, and guiding an intervention on the actual object based on the location or orientation of the marker with respect to the object in the perceived 3D image.

### BRIEF DESCRIPTION OF THE DRAWINGS

FIG. 1 is an illustration of an embodiment of the apparatus;

FIG. 2 is an illustration of a flowchart of a method of image-guided intervention according to the disclosed embodiments.

### DETAILED DESCRIPTION

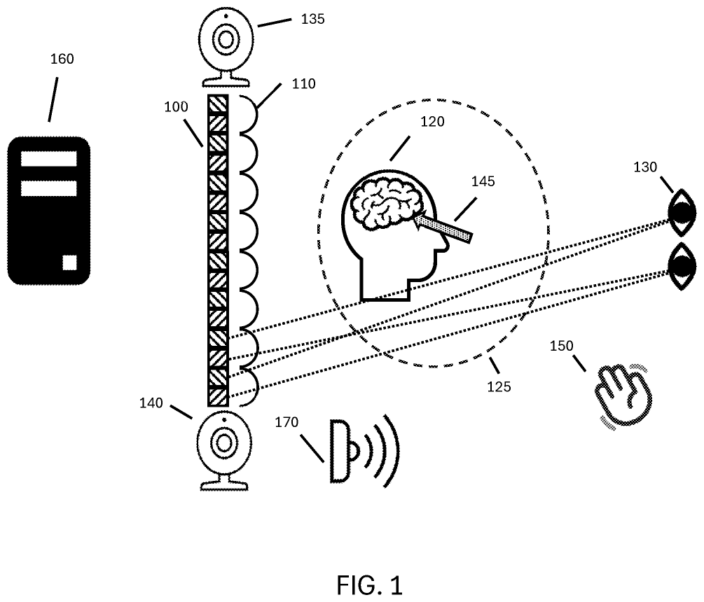

As shown in FIG. 1, the apparatus includes a display 100, one or more markers 145 and one or more cameras 140, 135. Components Light emitted by display 100 is modulated by a filter 110, which may be formed from lenticular or angular filters using mirrors as described by Otsubo or may be other modulators, and which forms a holographic display, e.g. a perceived image 120 of a 3D object in a region of space 125. The perceived image is formed in the eyes 130 of at least one viewer. One or more cameras 135, 140, or other monitors of electromagnetic radiation, sense the position and/or orientation of or the at least one viewer's eyes 130, hands 150 and markers 145. A computer 160 uses the information from the cameras 135, 140 to update the image on the display 100 based on the position of the eyes 130, head, hands 150, and markers 145 of the at least one viewer. The computer 160 may also utilize the one or more cameras 135, 140 in combination with additional types of sensors 170 such as LIDAR, ultrasound sensors, or structured light emitters to further improve the precision of tracking of the viewer's hands 150, as well as head and eye 130 positions.

The one or more cameras 135, 140 are capable of capturing images of a human viewer and record the position of the head, eyes 130, hands 150 as well as the position and orientation of a marker 145 that is placed by the human viewer in the perceived 3D object 120. The marker 145 may be a recognizable body part (a finger-tip, for example), a colored region, or may be an emitter of radiation. The emitted radiation may be detectable by the cameras 135, 140 and may not be visible to the human eye (for example, an infrared emitter). The marker 145 may be hand-held and contain switches or other haptic sensing elements for the viewer to confirm a location-based operation. Information from the cameras 135, 140 is provided to a computer 160 for analysis and segmentation to provide information about the position and orientation of the marker 145 with respect to the perceived 3D object 120. The computer 160 may adjust the images perceived by the viewer to include a representation of the marker 145 or of a visible representation of the intervention (for example, the electric field induced by a set of electromagnetic coils for neuromodulation). The viewer may choose to accept or reject or otherwise modify the intervention by moving or manipulating the marker 145 or other means.

In addition to tracking the position of the marker 145, the computer 160 also uses the cameras 135, 140 to track viewer's hands 150 and detect specific gestures that allow the viewer to manipulate the image. For example, a manipulation may be rotating the image in 3D space.

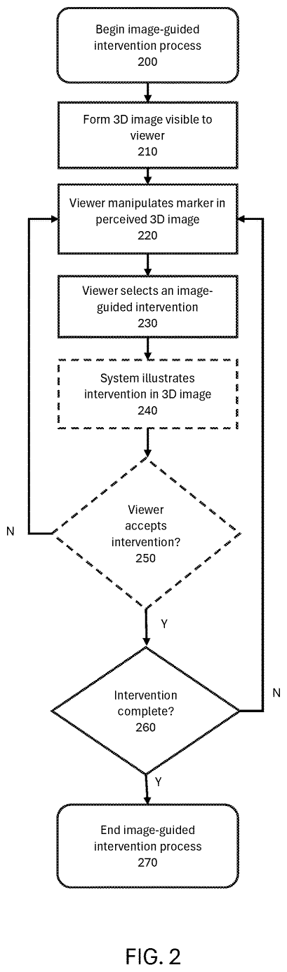

In a method of performing an image-guided intervention in FIG. 2, the user or viewer begins the image-guided intervention process 200. A computer presents images, for example of a brain tract, on monitors to form an image perceived by the viewer as a 3D object in space 210 (e.g. a holographic image). The viewer manipulates a marker within the perceived 3D object 220. The viewer (or another user working with the viewer) selects an image-guided intervention based on the position and/or orientation of the marker with respect to the perceived 3D object 230. In some embodiments, the image-guided intervention is the neuromodulation of the brain tract whose center is at the marker, the neuromodulation being effected by the application of an electric field along the direction of the orientation of the marker. In an optional operation 240, the computer modifies the image perceived by the viewer to illustrate the action that the intervention would do to the perceived 3D object. In an optional operation 250, the viewer (or affiliated user) accepts the selected intervention, and the intervention is carried out on the actual 3D object by the computer or by another connected computer. In some embodiments, the computer is connected to a device that applies electric fields to a tract in a brain for neuromodulation of that tract (e.g., multiple electrical pulses that activate the tract). The viewer (or another user working with the viewer) determines whether the intervention is complete 260. If so, the image-guided intervention is completed 270. If not, the user or viewer may repeat sections of the process. In some embodiments, the user is not queried in operation 250, and the intervention is carried out by the computer without the need for acceptance by the user.

In some embodiments, two cameras are used for the tracking. Artificial Intelligence or additional sensors 170 such as a LIDAR can be used to achieve similar functionality with only one camera. More than two cameras, additional sensors, and Artificial Intelligence can be used to improve the tracking precision.

In some embodiments, information from the cameras is used to adjust the disparity images which form the perceived 3D image.

In some embodiments, information from the at least two cameras is used to calibrate the system to increase positional accuracy.

In some embodiments, the perceived 3D image is a representation of an actual brain or brain tract anatomy generated by a magnetic resonance imaging system, and the viewer uses hands and hand gestures to select regions-of-interest in the perceived 3D image for a therapeutic intervention in the corresponding region of interest in the actual anatomy.

In some embodiments, the intervention may be neuromodulation delivered by a device using electropermanent magnets, as described in US patent publication 20220212022, entitled “METHOD AND APPARATUS FOR SELECTIVE MODULATION OF NEURONAL FUNCTION AND OF AND TISSUE PERMEABILITY WITH MRI CORRELATION”.

For the purposes of this disclosure, the term image-guided intervention means the actuation of a process which will have different outcomes depending on the location of the actuation. In the disclosed embodiments, the image-guided intervention included the delivery of neuromodulation under images provided by magnetic resonance imaging. The term also includes video games, in which a marker manipulated by a user is used to control avatars in the game. The term also includes combat scenarios in which the marker is held by the user to actuate a robot or drone.

Those skilled in the art will recognize, upon consideration of the above teachings, that the above exemplary embodiments and the computers may be based upon use of one or more programmed processors programmed with a suitable computer program. However, the disclosed embodiments could be implemented using hardware component equivalents such as special purpose hardware and/or dedicated processors. Similarly, general purpose computers, microprocessor-based computers, micro-controllers, optical computers, analog computers, dedicated processors, application specific circuits and/or dedicated hard wired logic may be used to construct alternative equivalent embodiments.

Moreover, it should be understood that control and cooperation of the above-described components may be provided using software instructions that may be stored in a tangible, non-transitory storage device such as a non-transitory computer readable storage device storing instructions which, when executed on one or more programmed processors, carry out the above-described method operations and resulting functionality. In this case, the term “non-transitory” is intended to preclude transmitted signals and propagating waves, but not storage devices that are erasable or dependent upon power sources to retain information.

Those skilled in the art will appreciate, upon consideration of the above teachings, that the program operations and processes and associated data used to implement certain of the embodiments described above can be implemented using disc storage as well as other forms of storage devices including, but not limited to non-transitory storage media (where non-transitory is intended only to preclude propagating signals and not signals which are transitory in that they are erased by removal of power or explicit acts of erasure) such as for example Read Only Memory (ROM) devices, Random Access Memory (RAM) devices, network memory devices, optical storage elements, magnetic storage elements, magneto-optical storage elements, flash memory, core memory and/or other equivalent volatile and non-volatile storage technologies without departing from certain embodiments. Such alternative storage devices should be considered equivalents.

While various exemplary embodiments have been described above, it should be understood that they have been presented by way of example only, and not limitation. Thus, the breadth and scope of the present invention should not be limited by any of the above-described exemplary embodiments but should instead be defined only in accordance with the following claims and their equivalents.

1. An apparatus for image-guided intervention comprising:
one or more cameras configured to capture images of a human viewer,
components for generating an aerial perceived 3D image of an actual object, and
at least one marker visible to the one or more cameras,
wherein information collected by one or more cameras is used to capture a position or orientation of the at least one marker, manipulatable by the human viewer, with respect to the object in the perceived 3D image.

2. The apparatus of claim 1, wherein the components include a computer and a light emitting display, and at the position or the orientation of the marker is used to direct the computer to carry out a therapeutic intervention.

3. The apparatus of claim 1, wherein the marker emits radiation that is not visible to the human eye.

4. The apparatus of claim 1, wherein the information collected by one or more cameras and information collected from additional sensors calibrates the apparatus.

5. The apparatus of claim 1, wherein the position or orientation of the marker is used to direct a therapeutic intervention to the actual object using neuromodulation.

6. The apparatus of claim 5, wherein the components modify the perceived 3D image to show the outcome a selected intervention has on the actual object.

7. The apparatus of claim 1, wherein the perceived 3D image is formed from information collected about the object with a system utilizing electropermanent magnets.

8. A method of image-guided intervention comprising:
generating an aerial perceived 3D image of an actual object based on images of a user captured by an apparatus with one or more cameras,
placing a marker within the perceived 3D image, and
guiding an intervention on the actual object based on the location or orientation of the marker with respect to the object in the perceived 3D image.

9. The method of claim 7, wherein the marker emits radiation that is not visible to the human eye.

10. The method of claim 7, wherein information collected by the one or more cameras and information collected by additional sensors is used to calibrate the apparatus.

11. The method of claim 7, further comprising: tracking hand gestures via the one or more cameras and via additional sensors to manipulate the perceived 3D image.

12. The method of claim 7, further comprising tracking what part of the perceived 3D image the user is looking at based information collected by one or more cameras and additional sensors is used to and updating the perceived 3D image based on the tracking.

13. The method of claim 7, wherein the perceived 3D image is formed from information collected with a system utilizing electropermanent magnets.

14. The method of claim 7, wherein the guiding comprises modifying the perceived 3D image to show the outcome intervention has on the actual object.
